(19) United States
(12) Patent Application Publication
(10) Pub. No.: US 2012/0303069 A1
(43) Pub. Date: Nov. 29, 2012

(54) RING AND BONE FIXATION SYSTEM

(75) Inventors: SHANG-CHIH LIN, Hsinchu (TW); YU-MING WANG, Hsinchu (TW)

(73) Assignee: UNITED ORTHOPEDIC CORPORATION, Hsinchu (TW)

(21) Appl. No.: 13/171,843

(22) Filed: Jun. 29, 2011

(30) Foreign Application Priority Data
May 26, 2011 (TW) 100209435

(51) Int. Cl. A61B 17/80

(52) U.S. Cl. 606/290

(57) ABSTRACT

The present invention discloses a ring, embedded in a bone plate for fixing the relative position of a bone screw and the bone plate. The ring has a sidewall and the sidewall has a plurality of gaps. The ring further comprises a lower holding portion and an upper holding portion, the lower holding portion and the upper holding portion are utilized to hold the ring on one of the surface of the bone plate and the corresponding surface thereof respectively. The present invention is capable of fixing the screw to the through hole of the bone plate in various directions or angles so as to prevent the bone screw from passing through the joint, the smashed area or the area of severe osteoporosis thereof. Furthermore, the present invention is easy to operate, capable of avoiding damage and decreasing the trouble for the operator thereof.

### BACKGROUND OF THE INVENTION

1. Field of the Invention

The present invention relates to a ring and a bone fixation system. More particularly, the present invention relates to a ring for fixing a relative position of a bone screw and a bone plate, and a bone fixation system utilizing the ring.

2. Description of the Prior Art

In prior art, for curing the bone fracture, the doctor will connect two broken bones by an operation and attach the bone plate near the broken bones. Then, the bone plate will be fixed with the bones by a bone screw passing through the threaded holes on the bone plate. Accordingly, the bone screw will press the broken bones to accelerate the healing time. Generally, the design of the fixing structure of the bone plate and the bone screw is that the bone screw can be fixed with the threaded holes of the bone plate. However, if the patient with osteoporosis fracture or crush fracture, the operation will become difficult. The bone screw only can be fixed with fixing angle, so that the bone screw may penetrate the joint and the risk is high. If the threaded holes are located below the smashed area or the area of severe osteoporosis, the inclination angle will be changed to avoid the smashed area or the area of severe osteoporosis. Thus, the difficulty of the operation will be increased. The inclination angle of the bone screw can be changed lower than 10 degrees, however, the threaded holes and the screw may be damaged. Accordingly, the bone screw will not be used again.

Take TW. patent No. M282644 for example, a scope of the said patent is to provide a fixing device structure for the bone fracture. The fixing device is capable of fixing the screw to the through hole of the bone plate in various directions or angles, so the fixing direction can be changed. Thus, the fixing position can be changed with wide range and the process of the operation will become smooth. By threaded holes with different angles, the bone screw can pass through the threaded holes to fix two broken bones. Accordingly, the fixing device structure is capable of avoiding damage and decreasing the trouble for the operator. However, Taiwan Patent No. M282644 discloses a design with the bottom side of the through hole, an adjustment slot and an angle glove with a hook-type tenon. Thus, the user shall determine the position and the direction of the pre-determined tenon and the adjustment slot, and applies a huge pressure by a tool, for letting the hook-type tenon of the angle glove be fixed in the through hole of the bone plate. Thus, jig devices or tools shall be developed. In the process, if the angle of the spiral angle of the angle glove is wrong and the user wants to remove the angle glove, the user shall use a tool to let the angle glove shrink. Accordingly, the angle glove with a hook-type tenon will be damaged easily.

To sum up, developing a fixation device for curing the bone fracture became an important issue in the field. The fixation device shall be capable of fixing the screw to the through hole of the bone plate in various directions or angles and prevent the bone screw from passing through the joint, the smashed area or the area of severe osteoporosis. Furthermore, the fixation device shall be easy to operate, capable of avoiding damage and decreasing the trouble for the operator.

### SUMMARY OF THE INVENTION

A scope of the invention is to provide a ring embedded in a bone plate, for fixing a relative position of a bone screw and the bone plate. The ring has a sidewall, the sidewall has a plurality of gaps. The ring further comprises a lower holding portion and a bottom surface. The lower holding portion is located at the junction of the sidewall and a bottom surface of the ring, for holding a lower surface of the bone plate. The lower holding portion is formed by extending outward from the sidewall. The upper holding portion is located at the junction of the sidewall and a top surface of the ring, for holding an upper surface of the bone plate, wherein the upper surface is relative to the lower surface. Wherein, the lower surface is contacted with a bone, the upper surface is relative to the lower surface. Additionally, the plurality of gaps extend from the bottom surface and toward the top surface of the ring along the normal vector of the bottom surface. The plurality of gaps are formed at the sidewall in a matrix form. The ring further comprises a penetrated hole, wherein a determined angle is formed between the extension direction of the penetrated hole and the horizontal direction of the bone plate. The determined angle is larger than 100 degrees or less than 80 degrees.

Another scope of the invention is to provide a bone fixation system. The bone fixation system comprises a bone plate, a bone screw and a ring. The bone plate comprises an upper surface and a lower surface. The lower surface is contacted with the bone, the upper surface is relative to the lower surface. The lower surface comprises a lower concave structure for holding the lower holding portion of the ring. The upper surface comprises an upper concave structure, for holding the upper holding portion of the ring. The lower concave structure and the upper concave structure pass through and connect to each other by a through hole, so that the ring can be embedded in the bone plate.

Compared to prior art, the bone fixation system lets the lower holding portion pass through the through hole of the bone plate and hold the lower concave structure of the bone plate by the elastic deformation of the sidewall of the ring. Additionally, the upper holding portion holds the upper concave structure so that the ting can be embedded in the bone plate. The ring can be embedded in the bone plate by a pressure applied from a tool easily. The ring can be prevented from damaging, so that the trouble of the medical personnel can be decreased. Furthermore, the bone screw can be fixed in the hole of the ring tightly and the ring is embedded in the bone plate, so that the bone plate can be fixed in the bone by the bone screw. The fixing angle can be changed by the different rings which have different determined angles. Thus, the fixing angle can be changed and the through hole of the bone plate can be fixed in various directions or angles. Accordingly, the doctor can choose the fitting angles match with bone screw for preventing the bone screw from passing through the joint, the smashed area or the area of severe osteoporosis.

The advantage and spirit of the invention may be understood by the following recitations together with the appended drawings.

### BRIEF DESCRIPTION OF THE APPENDED DRAWINGS

FIG. 1A illustrates parts of a cross-section schematic diagram of the lateral side of a bone fixation system according to an embodiment of the invention.

FIG. 1B illustrates parts of a cross-section schematic diagram of the front side of a bone fixation system according to an embodiment of the invention.

FIG. 2 illustrates a 3D schematic diagram of a ring according to an embodiment of the invention.

FIG. 3 illustrates a bottom view of a ring according to an embodiment of the invention.

FIG. 4 illustrates a top view of a ring according to an embodiment of the invention.

### DETAILED DESCRIPTION OF THE INVENTION

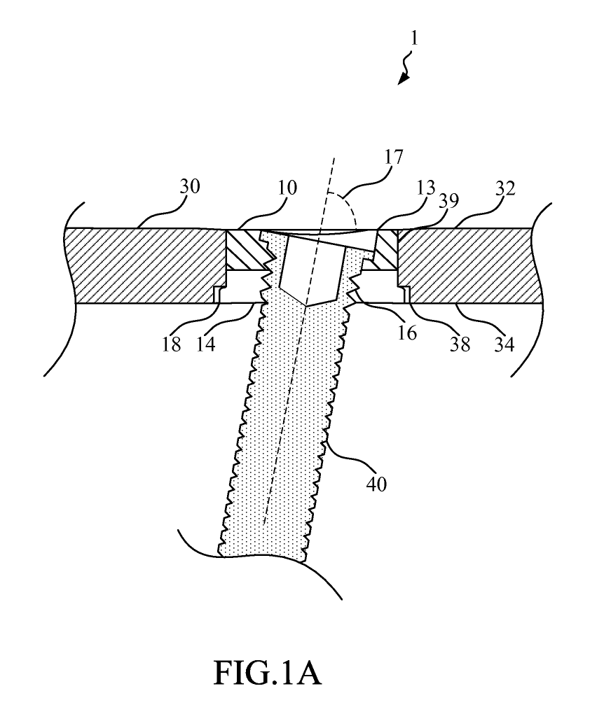

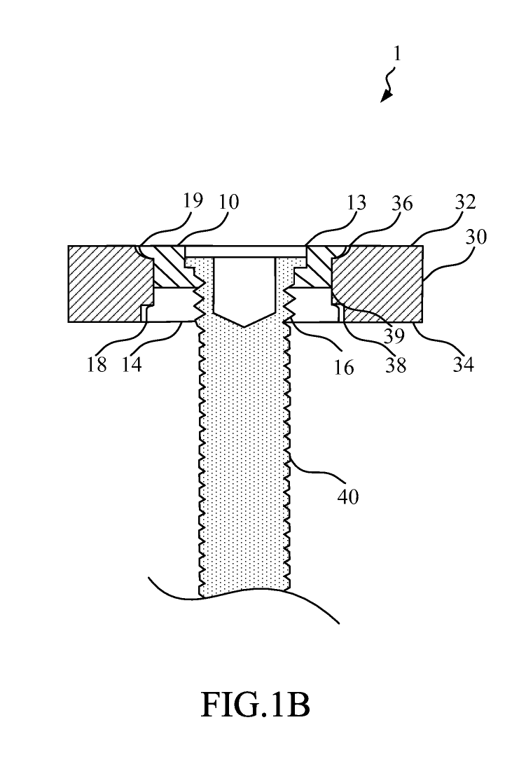

Please refer to FIG. 1A. FIG. 1A illustrates parts of a cross-section schematic diagram of the lateral side of a bone fixation system according to an embodiment of the invention. The invention is to provide a bone fixation system 1 for fixing a bone. The bone fixation system 1 comprises a bone plate 30, a bone screw 40 and a ring 10. The ring 10 is embedded in a bone plate 30, for fixing a relative position of a bone screw 40 and the bone plate 30. In practice, the bone screw 40 can be a screw made by biomedical materials. The biomedical materials comprise carbon fibers, metals, fiber reinforced plastics or other materials which are compatible in human bodies. The bone screw 40 penetrates the ring 10 and be fixed in the bone. At the same time, the ring 10 can fix the relative position of the bone screw 40 and the bone plate 30, so that the relative position of the bone screw 40, the ring 10 and the bone plate 30 can be fixed.

Please refer to FIG. 1B. FIG. 1B illustrates parts of a cross-section schematic diagram of the front side of a bone fixation system according to an embodiment of the invention. In the embodiment, the bone plate 30 comprises an upper surface 32 and a lower surface 34. The lower surface 34 is contacted with the bone. The upper surface 32 is relative to the lower surface 34. Additionally, the lower surface 34 comprises a lower concave structure 38 and the upper surface 32 comprises an upper concave structure 36. The lower concave structure 38 and the upper concave structure 36 pass through and connect to each other. In the embodiment, the lower concave structure 38 and the upper concave structure 36 pass through each other by a through hole 39. Furthermore, the ring 10 can be fixed in the bone plate 30 by the through hole 39. However, the invention can be, but not limited to the said design. The through hole 39 can be formed by the bone plate 30 connected to the upper concave structure 36 through the lower concave structure 38 for letting the ring 10 be embedded.

In the embodiment, the lower concave structure 38 is formed on the lower surface 34 of the bone plate 30. The concave area of the lower concave structure 38 is larger than the area of the through hole 39, wherein the lower concave structure 38 is an elliptical slot. However, the lower concave structure 38 is not limited to the said design, the lower concave structure 38 also can be a strip of slot or a concave structure corresponds to the shape of the through hole 39. The upper concave structure 36 is formed on the upper surface 32 of the bone plate 30. The concave area is larger than the area of the through hole 39. The concave area decreases and decreases along the extension direction of the lower surface 34, and a paraboloid is formed accordingly. Wherein, the concave shape of the upper concave structure 36 on the upper surface 32 is an ellipse. However, the lower concave structure 38 is not limited to the said design, the lower concave structure also can be a concave structure corresponds to the shape of the through hole 39 or the lower concave structure 38.

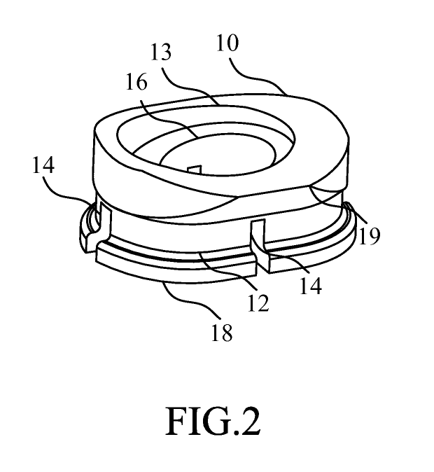

Please refer to FIG. 2. FIG. 2 illustrates a 3D schematic diagram of a ring according to an embodiment of the invention. The ring comprises a sidewall 12. The sidewall 12 is a ring structure and comprises a plurality of gaps 14. In the embodiment, the plurality of gaps 14 let the sidewall 12 has enough space to deform when the ring 10 is embedded in the bone plate 30.

Please refer to FIG. 1B and FIG. 2 again. In the embodiment, the ring 10 further comprises a lower holding portion 18 and an upper holding portion 19. The lower holding portion 18 is located at the junction of the sidewall 12 and a bottom surface of the ring 10, for holding a lower surface 34 of the bone plate 30. Wherein, the lower surface is contacted with the bone, the top surface is relative to the bottom surface. In the embodiment, the lower holding portion 18 is formed by extending outward from the sidewall 12 and divided into a plurality of intervals. Thus, the lower holding portion 18 will be a ring structure which is similar to the sidewall 12. The area surrounded by the lower holding portion 18 is larger than the area of the through hole 39, so that the lower holding portion 18 can hold and prevent the lower surface 34 from slipping. The upper holding portion 19 is also formed by extending outward from the sidewall 12, and the extension space of the upper holding portion 19 will become larger and larger toward the top surface of the ring 10. Thus, parts of the upper holding portion 19 will be a structure which is similar to a paraboloid. Wherein, the area surrounded by the upper holding portion 19 is larger than the area of the through hole 39, so that the upper holding portion 19 can hold and prevent upper surface 32 from slipping.

In the embodiment, the lower holding portion 18 passes through the through hole 39 and hold the lower concave structure 38 of the bone plate 30. The upper holding portion 19 holds the upper concave structure 36 of the bone plate 30.

Please refer to FIG. 1B again. In the embodiment, the ring 10 passes through the through hole 39 from the upper surface 32 of the bone plate 30. Additionally, the ring 10 let the lower holding portion 18 shrink inside, so that the area surrounded by the lower holding portion 18 will be smaller than the area of the through hole 39. Accordingly, the lower holding portion 18 passes through the through hole 39 from the upper surface 32 of the bone plate 30 and toward lower concave structure 38 located at the lower surface 34. Because the concave area of the lower concave structure 38 is larger than the through hole 39, the lower holding portion 18 has enough space to extend outward by the elastic deformation of the sidewall 12. Furthermore, before the lower holding portion 18 pass through the through hole 39, the area surrounded by the lower holding portion 18 is larger than the area of the through hole 39 before the lower holding portion 18 pass through the through hole 39. Thus, the lower holding portion 18 can hold the lower concave structure 38 by recovering the shape before the lower holding portion 18 passed through the through hole 39. Accordingly, the lower holding portion 18 can be prevented from penetrating the through hole 39 from the lower surface 34 of the bone plate 30, so that the ring will not slip from the bone plate 30. In addition, when the lower holding portion 18 pass through the through hole 39, the area surrounded by the upper holding portion 19 is larger than the area of the through hole 39, so that the upper holding portion 19 can hold and prevent upper surface 32 from slipping. Thus, the ring 10 can be embedded in the plate 30.

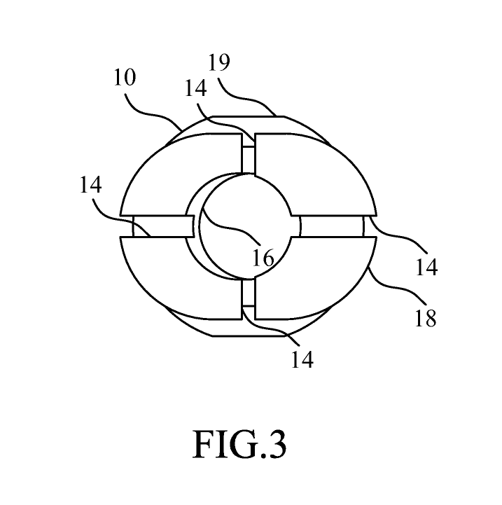

Please refer to FIG. 2 and FIG. 3. FIG. 3 illustrates a bottom view of a ring according to an embodiment of the invention. In the embodiment, the plurality of gaps extend from the bottom surface and toward the top surface of the ring along the normal vector of the bottom surface. That is to say, the plurality of gaps and the bottom surface of the ring 10 are vertical correspondingly. The plurality of gaps 14 are formed at the sidewall 12 in a matrix form. The matrix form means that the plurality of gaps 14 are evenly distributed at the sidewall 12. That is to say, the plurality of gaps 14 divides the lower holding portion 18 and the parts of the sidewall 12 near the lower holding portion 18 into a plurality of intervals. Accordingly, the sidewall can elastically deform easily.

In practice, because of the plurality of gaps 14, the sidewall 12 has enough space to elastically deform for decreasing the resistance generated by the extrusion of the material. Additionally, because the plurality of gaps 14 divide the sidewall 12 into many intervals, the sidewall 12 can elastically deform separately and decrease the shear stress can be decreased. Furthermore, because the stress of the material can be concentrate, the resistance of the elastic deformation will be decreased and the material can be prevented from damaging. Compared to prior art, the ring can be embedded in the bone plate by a pressure applied from a tool easily and prevented from damaging. Thus, the trouble of the medical personnel can be decreased.

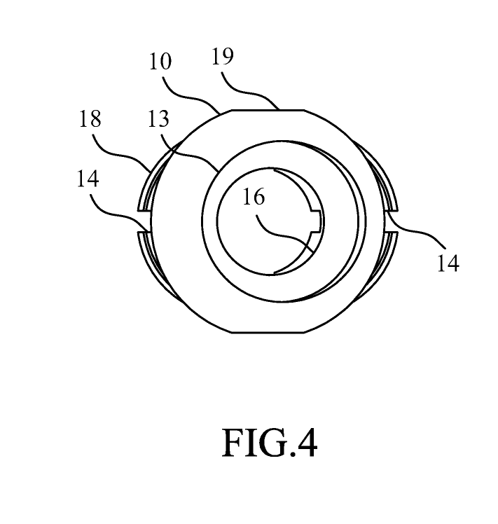

Please refer to FIG. 1 and FIG. 4. FIG. 4 illustrates a top view of a ring according to an embodiment of the invention. In the embodiment, the ring 10 comprises a penetrated hole 16. The penetrated hole 16 penetrates the top surface and the bottom surface of the ring 10. Additionally, the ring 10 further comprises an opening part 13. The opening part 13 is formed on the top surface of the ring 10. In practice, the penetrated hole 16 has a screw thread to let the bone screw 40 be engaged with the penetrated hole 16. The penetrated hole 16 is connected to the opening part 13. The bone screw 40 penetrates the ring 10 and be fixed in the bone through the penetrated hole 16 and the opening part 13. The screw thread is engaged with the bone screw 40 to let the bone screw 40 be fixed in the ring 10 tightly. Because the ring 10 is embedded and fixed in the bone plate, the bone plate 30 can be fixed on the bone. Furthermore, a determined angle 17 is formed between the extension direction of the penetrated hole 16 and the horizontal direction of the bone plate 30. In practice, the determined angle 17 can be larger than 100 degrees or less than 80 degrees. That is to say, the determined angle 17 can be 60 degrees, 70 degrees, 110 degrees or 120 degrees. Thus, the bone screw 40 can be fixed with different angles and not limited to right angle.

In practice, the fixing angle can be changed by the different rings which have different determined angles. Thus, the fixing angle can be changed and the through hole of the bone plate can be fixed in various directions or angles. Accordingly, the doctor can choose the fitting angles match with bone screw for preventing the bone screw from passing through the joint, the smashed area or the area of severe osteoporosis.

To sum up, the bone fixation system lets the lower holding portion pass through the through hole of the bone plate and hold the lower concave structure of the bone plate by the elastic deformation of the sidewall of the ring. Additionally, the upper holding portion holds the upper concave structure so that the ting can be embedded in the bone plate. The ring can be embedded in the bone plate by a pressure applied from a tool easily. The ring can be prevented from damaging, so that the trouble of the medical personnel can be decreased. Furthermore, the bone screw can be fixed in the hole of the ring tightly and the ring is embedded in the bone plate, so that the bone plate can be fixed in the bone by the bone screw. The fixing angle can be changed by the different rings which have different determined angles. Thus, the fixing angle can be changed and the through hole of the bone plate can be fixed in various directions or angles. Accordingly, the doctor can choose the fitting angles match with bone screw for preventing the bone screw from passing through the joint, the smashed area or the area of severe osteoporosis.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A ring embedded in a bone plate, for fixing a relative position of a bone screw and the bone plate, wherein the ring has a sidewall, the sidewall has a plurality of gaps.

2. The ring of claim 1, further comprising a lower holding portion located at the junction of the sidewall and a bottom surface of the ring, for holding a lower surface of the bone plate, wherein the lower surface is contacted with a bone.

3. The ring of claim 2 wherein the lower holding portion is formed by extending outward from the sidewall.

4. The ring of claim 1, further comprising an upper holding portion located at the junction of the sidewall and a top surface of the ring, for holding an upper surface of the bone plate, wherein the upper surface is relative to the lower surface.

5. The ring of claim 1, wherein the plurality of gaps extend from the bottom surface and toward the top surface of the ring along the normal vector of the bottom surface.

6. The ring of claim 1, wherein the plurality of gaps are formed at the sidewall in a matrix form.

7. The ring of claim 1, further comprising a penetrated hole, wherein a determined angle is formed between the extension direction of the penetrated hole and the horizontal direction of the bone plate.

8. The ring of claim 7, wherein the determined angle is larger than 100 degrees or less than 80 degrees.

9. A bone fixation system for fixing a bone, comprising:
a bone plate;
a bone screw; and
a ring embedded in a bone plate, for fixing a relative position of a bone screw and the bone plate, wherein the ring has a sidewall, the sidewall has a plurality of gaps.

10. The bone fixation system of claim 9, wherein the bone plate comprises an upper surface and a lower surface, the lower surface is contacted with the bone, the upper surface is relative to the lower surface, the lower surface comprises a lower concave structure, the upper surface comprises an upper concave structure, the lower concave structure and the upper concave structure pass through and connect to each other.

11. The bone fixation system of claim 10, wherein the ring comprises a lower holding portion located at the junction of the sidewall and a bottom surface of the ring, for holding a lower surface of the bone plate.

12. The bone fixation system of claim 11, wherein the lower holding portion is formed by extending outward from the sidewall.

13. The bone fixation system of claim 11, wherein the lower holding portion further holds the lower concave structure.

14. The bone fixation system of claim 10, wherein the ring comprises an upper holding portion located at the junction of the sidewall and a top surface of the ring, for holding an upper surface of the bone plate.

15. The bone fixation system of claim 14, wherein the upper holding portion further holds the upper concave structure.

16. The bone fixation system of claim 10, wherein the plurality of gaps extend from the bottom surface and toward the top surface of the ring along the normal vector of the bottom surface.

17. The bone fixation system of claim 10, wherein the plurality of gaps are formed at the sidewall in a matrix form.

18. The bone fixation system of claim 10, wherein the ring comprises a penetrated hole, a determined angle is formed between the extension direction of the penetrated hole and the horizontal direction of the bone plate.

19. The bone fixation system of claim 18, wherein the determined angle is larger than 100 degrees or less than 80 degrees.
